(19) United States
(12) Patent Application Publication
(10) Pub. No.: US 2025/0099496 A1
(43) Pub. Date: Mar. 27, 2025

(54) Dihydronicotinamide Riboside (NRH) Therapy for Osteoarthritis and other Conditions

(71) Applicants: The Regents of the University of California, Oakland, CA (US); University of South Alabama, Mobile, AL (US)

(72) Inventors: Ru Bryan, La Jolla, CA (US); Marie Migaud, Mobile, AL (US)

(21) Appl. No.: 18/730,598

(22) PCT Filed: Jan. 19, 2023
(86) PCT No.: PCT/US2023/011109

§ 371 Date: Jul. 19, 2024

Related U.S. Application Data
(60) Provisional application No. 63/300,950, filed on Jan. 19, 2022.

(51) Int. Cl. A61K 31/706; A61K 31/519; A61K 31/7068; A61P 19/02; A61P 19/06

(52) U.S. Cl. CPC A61K 31/706; A61K 31/519; A61K 31/7068; A61P 19/02; A61P 19/06

(57) ABSTRACT

Compositions and methods of treatment for osteoarthritis and cartilage damage in a subject comprising administering to a subject in need thereof an effective amount of a dihydronicotinamide riboside (NRH). Compositions and methods of treatment for osteoarthritis, cartilage damage, gout or aging-associated diseases in a subject comprising administering to a subject in need thereof an effective amount of NRH and/or nicotinamide riboside (NR) and administering to the subject an effective amount of a purine nucleoside phosphorylase (PNP) inhibitor.

### CROSS REFERENCE TO RELATED APPLICATIONS

The present application claims the priority benefit of U.S. Provisional Application No. 63/300,950 filed on Jan. 19, 2022, the entire contents of which are hereby incorporated by reference herein.

### TECHNICAL FIELD

The present invention relates to treatments for osteoarthritis and other conditions.

### BACKGROUND

Joint injury and aging are the primary risk factors for development of osteoarthritis (OA), the most common form of arthritis. Currently, there are not yet effective medical therapies to prevent or slow the disease process in OA. Thus, developing effective OA disease-modifying medical therapy is a major unmet medical need. Progressive degeneration of articular cartilage is a core OA disease feature. Chondrocytes, the sole cells in articular cartilage, are responsible for maintaining the homeostatic balance between extracellular matrix anabolism and catabolism. In OA, chondrocytes undergo metabolic alterations triggering changes in energy metabolism due to altered bioenergy sensing, leading to chondrocyte dysfunction. This is amplified by local inflammatory processes, which promote excessive chondrocyte catabolic responses by inducing expression and activities of matrix degrading enzymes such as matrix metalloproteinases (MMPs).

Nicotinamide adenine dinucleotide (NAD⁺), a key intermediate metabolite, serves as a cofactor for numerous enzymes involved in cellular energy metabolism. It modulates adaptive responses of cells to bioenergetic and oxidative stress, and critically regulates multiple cell-signaling pathways. Mounting evidence indicates that maintenance of intracellular NAD⁺ content is critical for tissue homeostasis. Changes in NAD⁺ levels are related to the balance between NAD⁺ biosynthesis and degradation. Most cellular NAD⁺ is synthesized through salvage pathways from nicotinamide (NAM) or from NAD⁺ precursors (e.g., nicotinamide riboside (NR)). Another NAD⁺ precursor dihydronicotinamide riboside (NRH), a reduced form of NR, uses a newly identified pathway for NAD biosynthesis. Cellular NAD⁺ levels decline with aging, associated with increased expression of CD38, the main NADase in mammalian tissues contributing substantially to NAD degradation. Inflammation can cause NAD⁺ decline due to activation of consumers of NAD⁺ such as CD38.

### SUMMARY OF THE INVENTION

The disclosure provides, in embodiments, compositions and methods of treatment, including prevention, of osteoarthritis and cartilage injury and cartilage degradation in a subject comprising administering to a subject in need thereof an effective amount of dihydronicotinamide riboside (NRH). The invention contemplates that the administration can be of a pharmaceutical composition comprising NRH.

In embodiments, the invention provides that the administration increases NAD levels in articular chondrocytes of the subject. In embodiments, the invention provides that the administration inhibits progression of osteoarthritis. In embodiments, the invention provides that the administration inhibits expression of matrix-degrading enzymes.

In embodiments, the invention provides pharmaceutical compositions comprising dihydronicotinamide riboside (NRH) and one or more pharmaceutically acceptable excipients, to treat osteoarthritis, cartilage injury, or cartilage degradation in a subject. In embodiments, the invention provides that the administration is oral or parenteral.

In embodiments, the invention provides that the method further comprises administering to the subject an effective amount of a purine nucleoside phosphorylase (PNP) inhibitor. In embodiments, the invention provides that the PNP inhibitor administration occurs before the NRH administration.

In embodiments, the invention provides a method of treatment for gout in a subject comprising administering to a subject in need thereof an effective amount of a purine nucleoside phosphorylase (PNP) inhibitor, and administering to the subject an effective amount of dihydronicotinamide riboside (NRH).

In embodiments, the PNP inhibitor administration occurs before the NRH administration. In embodiments, the treatment increases NAD levels in the subject. In embodiments, the treatment inhibits expression of matrix-degrading enzymes. In embodiments, the treatment inhibits progression of gout.

In embodiments, the invention provides a method of treatment for aging-associated disease comprising administering to a subject in need thereof an effective amount of a purine nucleoside phosphorylase (PNP) inhibitor, and administering to the subject an effective amount of dihydronicotinamide riboside (NRH).

In embodiments, the PNP inhibitor administration occurs before the NRH administration. In embodiments, the treatment increases NAD levels in the subject. In embodiments, the treatment inhibits expression of matrix-degrading enzymes. In embodiments, the treatment inhibits progression of aging-associated disease. In embodiments, the aging-associated disease is selected from cardiovascular disease, neural and cognitive function disease, metabolic disease, muscular disease and kidney disease.

### BRIEF DESCRIPTION OF THE DRAWINGS

FIGS. 1A-1H show increased CD38 expression in chondrocytes is associated with reduced NAD/NAD ratio and increased catabolic activities.

FIGS. 2A-2G show treatment of OA chondrocytes with NRH raised cellular NAD levels and attenuated IL-1b-induced CD38 expression.

FIGS. 3A-3G show anti-catabolic effect of NRH.

FIGS. 4A-4F show oral supplementation of NRH limited OA development and associated pain in a post-traumatic OA model in mice.

FIG. 5 shows how NRH uses both the NRH-NMNH-NADH-NAD pathway and the NRH-NR-NMN-NAD pathway for NAD biosynthesis. The enzyme known to oxidize NRH to NR is called NQO2.

FIGS. 6A-6B show that the increased effectiveness of NR was due to inhibition of intracellular PNP.

FIGS. 7A-7B show the effectiveness of combination of low-dose of NAD precursors (NR or NRH) with a PNP inhibitor Ulodesine on inhibition of IL-1β-induced nitric oxide (NO) generation in human chondrocytes under the serum-presence condition.

FIG. 8 shows MSU crystals induced a robust increase in infiltrating inflammatory cells, which was significantly inhibited by either NR or NRH, indicating the capability of NR and NRH in reducing acute gouty inflammation.

### DETAILED DESCRIPTION

The present disclosure relates, in part, to compositions and methods of treatment for osteoarthritis, cartilage injury, cartilage degradation, and related conditions.

In embodiments, the invention provides methods of treatment, including prevention, of osteoarthritis, cartilage injury and/or cartilage degradation in a subject comprising administering to a subject in need thereof an effective amount of dihydronicotinamide riboside (NRH). The invention contemplates that the administration can be of a pharmaceutical composition comprising NRH.

In embodiments, the invention provides that the administration increases NAD levels in articular chondrocytes of the subject. In embodiments, the invention provides that the administration inhibits progression of osteoarthritis. In embodiments, the invention provides that the administration inhibits expression of matrix-degrading enzymes.

In embodiments, the invention provides pharmaceutical compositions comprising dihydronicotinamide riboside (NRH) and one or more pharmaceutically acceptable excipients, to treat osteoarthritis, cartilage injury, or cartilage degradation in a subject. In embodiments, the invention provides that the administration is oral or parenteral.

In embodiments, the invention provides that the method further comprises administering to the subject an effective amount of a purine nucleoside phosphorylase (PNP) inhibitor. In embodiments, the invention provides that the PNP inhibitor administration occurs before the NRH administration.

In embodiments, the invention provides a method of treatment for gout in a subject comprising administering to a subject in need thereof an effective amount of a purine nucleoside phosphorylase (PNP) inhibitor, and administering to the subject an effective amount of dihydronicotinamide riboside (NRH).

In embodiments, the PNP inhibitor administration occurs before the NRH administration. In embodiments, the PNP inhibitor is forodesine or ulodesine, or pharmaceutically acceptable salts thereof. In embodiments, the treatment increases NAD levels in the subject. In embodiments, the treatment inhibits expression of matrix-degrading enzymes. In embodiments, the treatment inhibits progression of gout.

In embodiments, the invention provides a method of treatment for aging-associated disease comprising administering to a subject in need thereof an effective amount of a purine nucleoside phosphorylase (PNP) inhibitor, and administering to the subject an effective amount of dihydronicotinamide riboside (NRH).

In embodiments, the PNP inhibitor administration occurs before the NRH administration. In embodiments, the PNP inhibitor is forodesine or ulodesine, or pharmaceutically acceptable salts thereof. In embodiments, the treatment increases NAD levels in the subject. In embodiments, the treatment inhibits expression of matrix-degrading enzymes. In embodiments, the treatment inhibits progression of aging-associated disease. In embodiments, the aging-associated disease is selected from cardiovascular disease, neural and cognitive function disease, metabolic disease, muscular disease and kidney disease.

Various further aspects and embodiments of the disclosure are provided by the following description.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention may employ conventional techniques of molecular biology (including recombinant techniques), microbiology, cell biology, biochemistry and immunology, which are within the skill of the art. Such techniques are explained fully in the literature, such as Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In embodiments, the disclosure provides pharmaceutical compositions comprising dihydronicotinamide riboside (NRH) and one or more pharmaceutically acceptable excipients or diluents. In embodiments, the disclosure provides pharmaceutical compositions comprising a PNP inhibitor and one or more pharmaceutically acceptable excipients or diluents. In embodiments, the PNP inhibitor can be selected from those known in the art, such as but not limited to ulodesine or forodesine, and pharmaceutically acceptable salts thereof, and other PNP inhibitors known and discovered in the future.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which dihydronicotinamide riboside (NRH) or the PNP inhibitor of the disclosure, such as but not limited to ulodesine or forodesine, and pharmaceutically acceptable salts thereof, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In embodiments, the NRH and the PNP inhibitor are administered to a subject as separate entities separately, wherein such administration provides therapeutically effective levels of the two compounds in the body of the patient. In embodiments, the PNP inhibitor is forodesine or ulodesine, or pharmaceutically acceptable salts thereof. In embodiments, the PNP inhibitor is administered to a subject before the NRH administration. In embodiments, the PNP inhibitor is administered to a subject from between 1 to 12 hours, or about 6 hours, before the NRH administration.

In some embodiments, the disclosure provides compositions and methods of treating or preventing a disease or disorder in a subject in need thereof, such as osteoarthritis and cartilage injury or degradation, comprising administering the NRH of the disclosure to the subject. In some embodiments, the disclosure provides compositions and methods of treating or preventing a disease or disorder in a subject in need thereof, such as osteoarthritis, cartilage injury or degradation, inflammatory arthritis, gout, aging-associated diseases, such as cardiovascular disease, neural and cognitive function disease, metabolic disease, muscular disease and kidney disease.

In embodiments, the NRH is administered to a subject in a dosage in the range of less than 400 mg/kg, or between 1-100 mg/kg, or 20-80 mg/kg or about 40-50 mg/kg. Dosages will vary within the ordinary skill of those in the art depending upon the age and condition of the subject. In embodiments, the NRH dosage is administered to a subject over a period of between 1 and 10 days, or more.

In embodiments, the PNP inhibitor, such as but not limited to ulodesine or forodesine, and pharmaceutically acceptable salts thereof, is administered to a subject in an oral dosage in the range of between 0.1-20 mg/kg, or 0.2-10 mg/kg or about 5 mg/kg. Dosages will vary within the ordinary skill of those in the art depending upon the selected PNP inhibitor, the route of administration, and the age and condition of the subject. Current clinical trials dosages for ulodesine are described at sec.gov/Archives/edgar/data/882796/000119312512311267/d383732dex991.htm. In embodiments, the PNP inhibitor is administered to a subject over a period of between 1 and 10 days, or more.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal in need of being treated by the methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject. As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” is encompassed by “treatment” and may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

The pharmaceutical compositions of the disclosure may be administered in a number of ways depending upon whether local or systemic treatment is desired. The pharmaceutical compositions are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intradermal, intrasynovial injection or infusions. In some embodiments, the pharmaceutical compositions of the present disclosure comprise intravenous administration.

### Examples

### NRH as a Novel Therapeutic Agent for Osteoarthritis Treatment

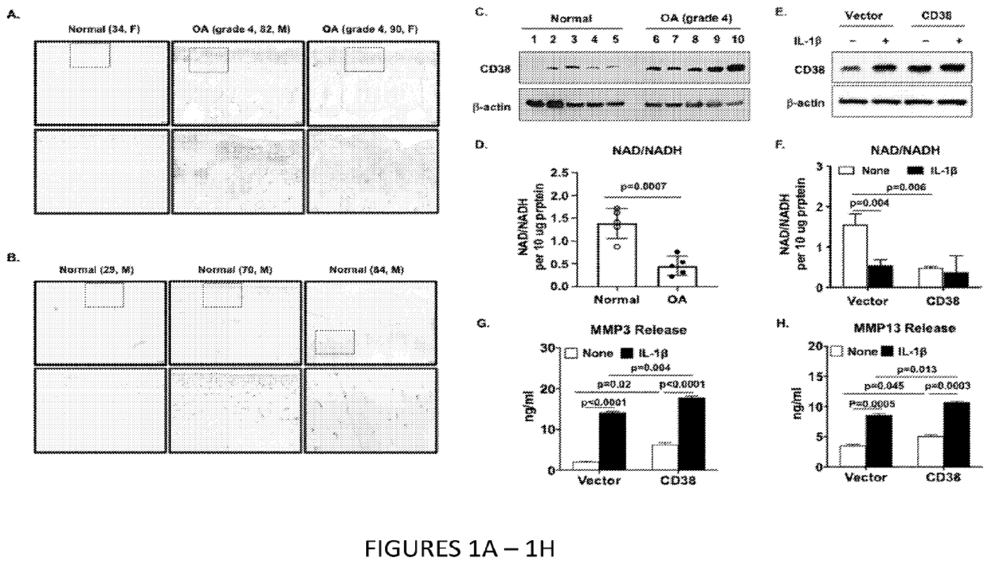

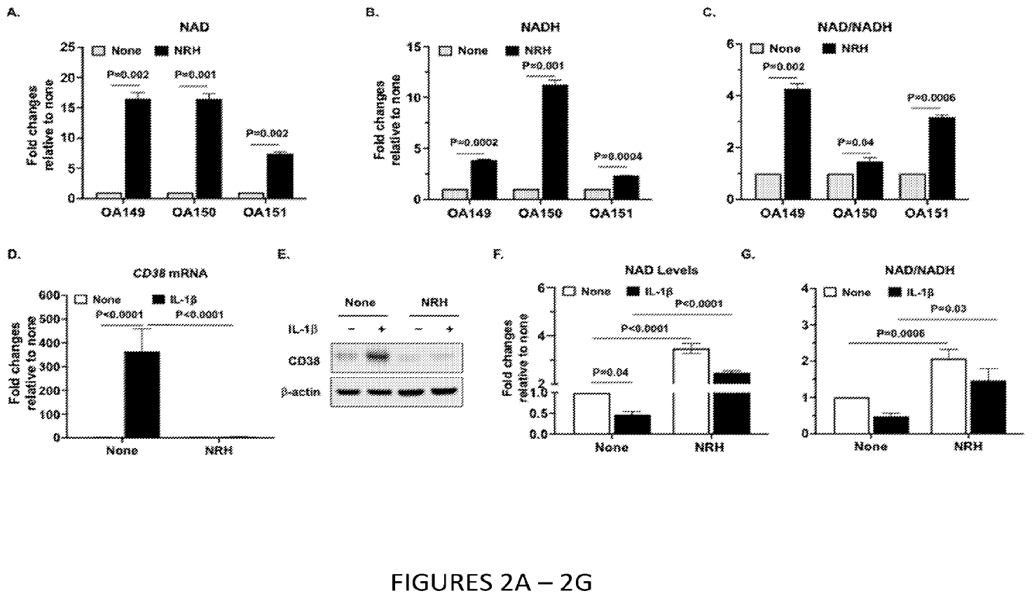

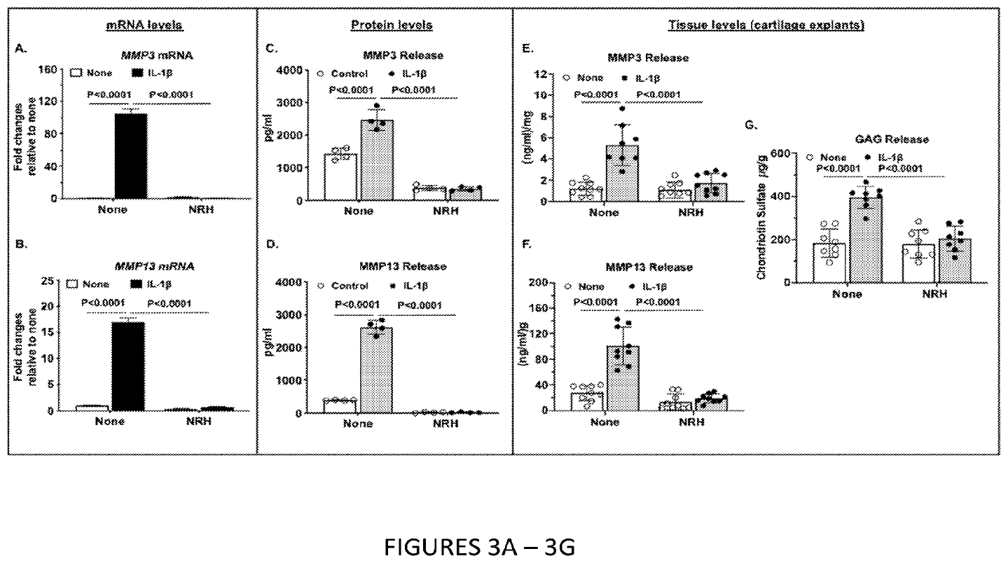

In this study, it was observed that CD38 expression is increased in human knee cartilage of both osteoarthritis (OA) and aged donors (FIGS. 1A and 1B), in primary human knee OA chondrocytes (FIG. 1C) and in chondrocytes stimulated with IL-1, a pro-inflammatory cytokine implicated to play an important role in OA pathogenesis (FIG. 1E). Chondrocytes with increased CD38 expression exhibit significant decrease in NAD/NADH ratio (FIGS. 1D and 1F) and increased expression of MMP3 and MMP13 (FIGS. 1G and 1H). Stimulation of human OA chondrocytes with NRH dramatically increased cellular levels of NAD, NADH and NAD/NADH ratio (FIGS. 2A, 2B, 2C). In addition, NRH prevented IL-1β-induced CD38 expression at both mRNA and protein levels (FIGS. 2D and 2E) and decline of NAD levels and NAD/NADH ratio (FIGS. 2F and 2G). Moreover, NRH completely attenuated IL-1β-induced expression of MMP3 and MMP13 at cellular (mRNA and protein) levels (FIGS. 3A, 3B, 3C, 3D) and tissue levels (FIGS. 3E and 3F). Importantly, NRH significantly inhibited IL-1β-induced release of matrix protein glycosaminoglycan (GAG, FIG. 3G), suggesting that NRH can inhibit cartilage matrix degradation. It was observed that NRH has higher potency than NR to inhibit IL-1β-induced catabolic responses in chondrocytes, evidenced by that similar efficacy was achieved by NRH used at concentration 10 times less than NR (data not shown).

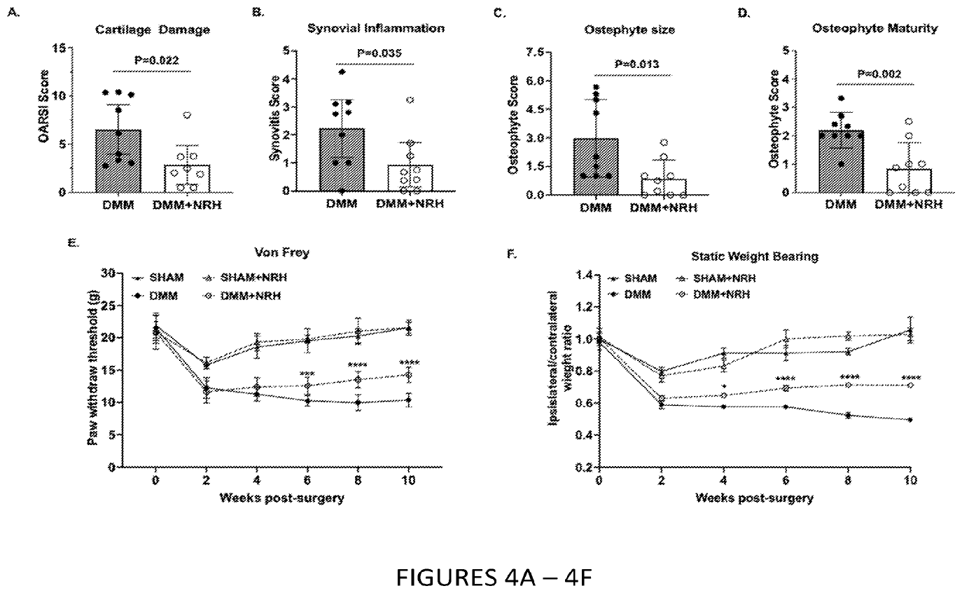

Given that NRH is a natural NAD precursor with oral bioavailability and its ability to not only promote NAD⁺ biosynthesis, but also prohibit NAD⁺ degradation by inhibiting CD38, it was next tested whether oral administration of NRH limits OA development in mice using a post-traumatic OA model. Destabilization of medial meniscus (DMM) surgery was performed on the right knee of mice to cause joint injury and started NRH treatment through gavage 2 week after the surgery. Pain behaviors such as pain sensitivity and spontaneous pain were evaluated by Von Frey and static weight bearing tests, respectively, at baseline and every 2 weeks after the surgery. At 10 weeks after the surgery, mice were sacrificed, and knee OA pathological changes including cartilage degradation, synovial inflammation and osteophyte formation were assessed. As shown in FIGS. 4A-4F, mice received NRH treatment exhibited significantly reduced cartilage damage (FIG. 4A), synovial inflammation (FIG. 4B) and osteophyte size and maturity (FIGS. 4C and 4D). In addition, mice received NRH treatment exhibited improved pain behaviors, evidenced by significantly increased paw withdraw threshold (Von Frey) and weight bearing capacity at 6, 8 and 10 weeks after the surgery (FIGS. 4E and 4F). These results suggest that NRH has OA disease modifying ability by limiting OA development and associated pain in mice.

Taken together, the data from this study implicate NRH as a novel therapeutic agent for treating and preventing OA. Preventing intracellular NAD+ content decline and/or restoration of NAD+ levels through NRH supplementation is a new approach to suppress cartilage degradation and OA development.

### Boosting Efficacy of NRH Through Inhibition of Purine Nucleoside Phosphorylase (PNP)

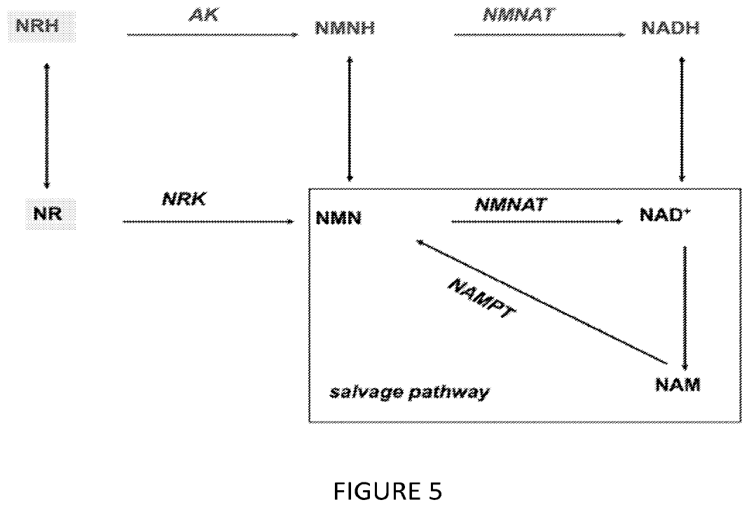

Both nicotinamide riboside (NR) and NRH are the NAD precursors. NR is found to be readily metabolized by the purine nucleoside phosphorylase (PNP) and is rapidly degraded to nicotinamide (NAM) in plasma (1,2), suggesting that the degradation of NR could compromise the ability of intact NR to reach peripheral tissues. Recent findings reveal that combining pharmacological inhibition of PNP by Immucillin H (also named as Forodesine) with NR supplementation in mice strongly diminished NR degradation to NAM, resulting in maintenance of high levels of NR in blood, kidney, and liver (3). NR, which is the oxidized form of NRH, is detectable in circulation after NRH treatment at much higher levels than those detected after NR administration (4). This indicates that NRH uses not only the NRH-NMNH-NADH-NAD pathway but also the NRH-NR-NMN-NAD pathway for NAD biosynthesis (see FIG. 5).

This example explored whether combining NRH with a PNP inhibitor can increase efficiency of NRH for NAD biosynthesis by preventing degradation of NR (oxidized from NRH) by the PNP. NRH, when combined with the PNP inhibitors, may have better efficacy than NRH alone, in limiting osteoarthritis development and progression. Evidence from in vitro studies demonstrates that combination of NAD precursors (NR or NRH) and the PNP inhibitors (e.g., Ulodesine or Forodesine) exerts synergistic effect on inhibition of chondrocyte responses to IL-1β, a proinflammatory cytokine that is implicated to participate in osteoarthritis (OA) pathogenesis.

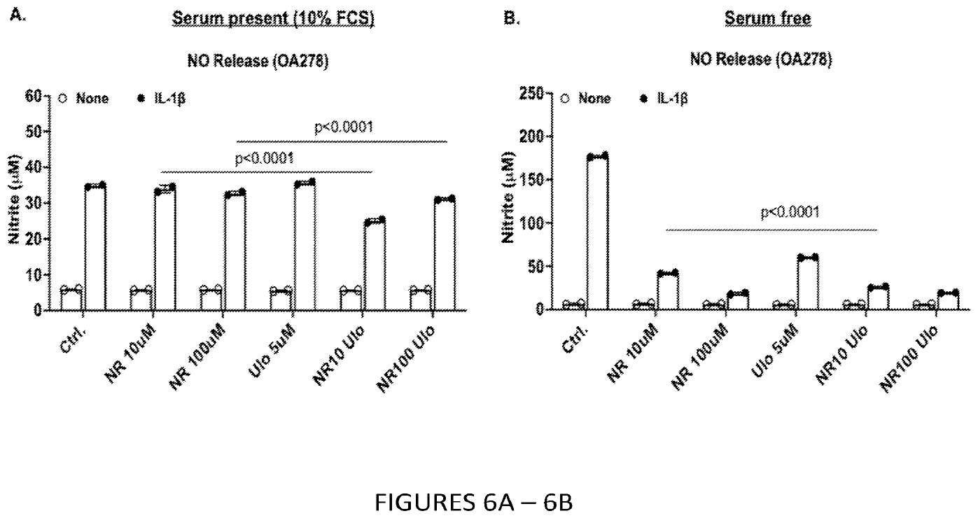

The effectiveness of NR on inhibition of IL-1β-induced nitric oxide (NO) generation in human chondrocytes was influenced by the presence or absence of serum (which contains PNP) in the cell culture media. Human OA chondrocytes (donor OA278) were treated with a PNP inhibitor Ulodesine (5 uM) for 2 h and then NR (10 or 100 uM) for 1 h, followed by IL1β (2 ng/ml) for 24 h under serum (10% FCS)-present (FIG. 6A) or serum-free condition (FIG. 6B). The conditioned media was used for measuring generation of nitric oxide (NO), a known contributor of cartilage degradation.

After being imported into the cells, NR is predominately metabolized by PNP (3). Since PNP is also present in the serum in the culture media, exogenous NR added to the media could also be metabolized. As shown in FIGS. 6A-6B, NR at 10 and 100 um concentration showed little effect under the serum-present condition (FIG. 6A) but exhibited robust inhibition under the serum-free condition (FIG. 6B) on IL-1β-induced NO release. This result suggests that the presence of PNP in the serum may cause rapid degradation of exogenous NR, therefore hindering NR function. Indeed, combination of NR (10 and 100 uM) with Ulodesine (5 uM) significantly inhibited IL-1β-induced NO release under the serum-present condition (FIG. 6A, p<0.0001). Notably, combination of NR (10 uM) with Ulodesine (5 uM) exhibited better inhibition of IL-1β-induced NO release, compared to NR (10 um) alone under the serum-free condition (FIG. 6B, p<0.0001), indicating that the increased effectiveness of NR was due to inhibition of intracellular PNP by Ulodesine.

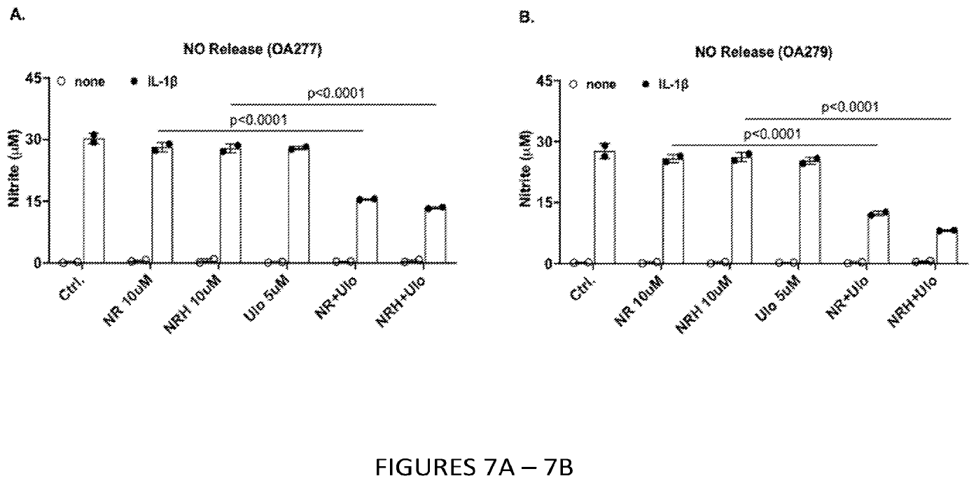

Effectiveness of combination of low-dose of NAD precursors (NR or NRH) with a PNP inhibitor Ulodesine on inhibition of IL-1β-induced nitric oxide (NO) generation in human chondrocytes under the serum-presence condition. Human OA chondrocytes from donor OA277 (FIG. 7A) or OA279 (FIG. 7B) were treated with Ulodesine (5 uM) for 2 h and then NR or NRH at 10 uM for 1 h, followed by IL1p (2 ng/ml) for 24 h under serum-present (10% FCS) condition. The conditioned media was used for measuring NO generation.

As seen in FIGS. 7A and 7B, either NR or NRH at low dose (10 uM) showed little effect on IL-1β-induced NO release. However, when they were combined with Ulodesine (5 uM), IL-1β-induced NO release were markedly inhibited (p<0.0001). These data suggest that inhibition of PNP may significantly enhance the stability and availability of NR or NRH, therefore increasing their effectiveness. It was previously demonstrated that significant inhibition of IL-1β-induced NO release in chondrocytes by NR or NRH alone was only observed when they were used at 200 uM. It seemed that low dose of NR or NRH, when combined with a PNP inhibitor, was almost as effective as high dose of NR or NRH used alone.

This finding suggest that one can lower the dose of NR or NRH by at least 10-fold to achieve their therapeutic effects with the aid of a PNP inhibitor. Also, the dose of PNP inhibitors could also be reduced, because both Forodesine and Ulodesine inhibit PNP with IC50 in the nM concentration range.

Comparing FIGS. 6A-6B to FIGS. 7A-7B, one can observe that combination of Ulodesine and NR if added sequentially to the cells has better inhibitory effect than added together at the same time. This suggests that inhibition of the PNP first by Ulodesine could have a better protection of NR from degradation because of presence of less PNP.

Broader Application of Combination of NR or NRH with the PNP Inhibitors

NR effects have been and/or are being investigated in many clinical trials in aging and age-related diseases including cardiovascular diseases, neural and cognitive functions, metabolic disturbances, and muscular and kidney injuries. Recent studies demonstrated that chronic oral supplementation of NR is well-tolerated, safe, and boosted human NAD⁺ metabolism in a dose-dependent manner (5-7). However, the clinical trial data showed that dietary NR supplementation has only mild benefits on blood pressure and exercise performance in elderly individuals (8) and has no significant impact on skeletal muscle mitochondria in obese and insulin-resistant men (9), as well as glucose tolerance and j-cell secretory capacity in nondiabetic men with obesity (10). Since NR is readily metabolized by the PNP, the short stability of NR in circulation may limit its effectiveness in a clinical application.

A combination of NR and the PNP inhibitors can overcome this limitation by increasing stability and bioavailability of NR. Comparing to NR, NRH is more stable and is detectable in circulation in mice after NRH treatment. As mentioned earlier, NR is detected at much higher levels than those detected after NR administration. NRH can use not only the NRH-NMNH-NADH-NAD pathway, but also the NRH-NR-NMN-NAD pathway, for NAD biosynthesis. Combining NRH with the PNP inhibitors can also increase efficiency of NRH at least partly by preventing degradation of NR oxidized from NRH. Given that low dose of NR or NRH, when combined with a PNP inhibitor, may achieve similar effects to high dose of NR or NRH used alone, long-term use of combination of low dose NR or NRH and a PNP inhibitor may significantly reduce the side effects if any when NR or NRH used at high dose.

Gout is a common inflammatory arthritis due to the deposition of monosodium urate (MSU) crystals in joints and soft tissues. It is associated with significant disease burden due to frequent painful flares and chronic inflammation. Gouty arthritis is a major and increasing public health problem given the substantial rise over the last two decades, along with increases in its severity, treatment complexity and resistance to treatment. Treatments for gout are focused on gout flare vs. those targeting urate lowering for managing hyperuricemia. Medications used for the treatment of gout flares such as glucocorticoids, non-steroidal anti-inflammatory drugs (NSAIDs) or colchicine, are relatively or absolutely contraindicated in many gout patients. More options are needed to expand the current urate-lowering drugs including xanthine oxidase inhibitors, uricosurics, and uricase.

Ulodesine has been tested in clinical trials for gout, as it blocks uric acid generation by inhibiting the PNP that produces hypoxanthine which is then converted to urate by xanthine oxidase. A randomized placebo-controlled trial compared doses of 40, 80 and 120 mg per day in 60 gout patients with baseline serum urate (SU)≥8 mg/dL (11, 12). In each arm approximately one third of participants achieved the target SU of less than 6 mg/dL.

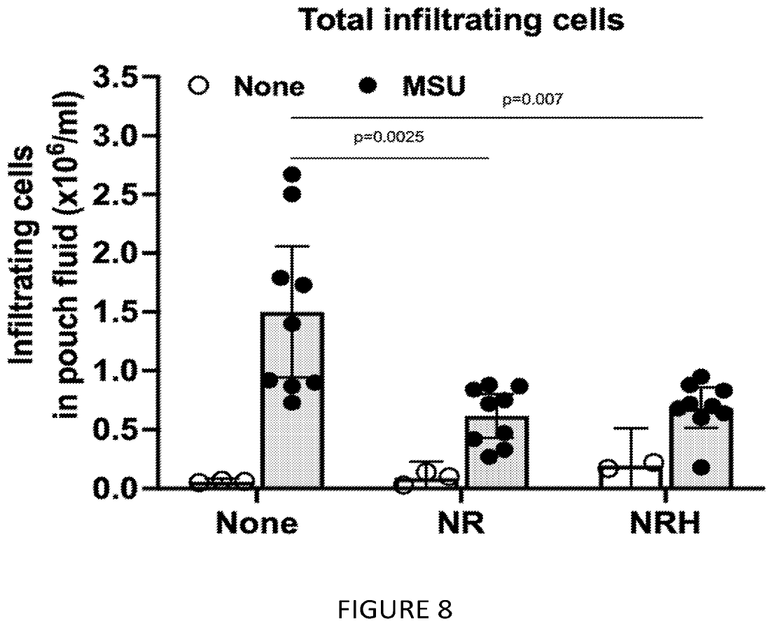

It was observed that blood NAD levels decrease, correlated with increase in CD38 expression in peripheral blood mononuclear cells in gout patients compared to healthy subjects. This suggests that boosting NAD levels with its precursor has therapeutic potential for controlling gouty flares. The effect of oral supplementation of NR or NRH (via gavage, 400 mg/kg/day) on MSU crystal-induced inflammation in the gout-like air pouch model in mice was tested (FIG. 8). The development of the air pouch leads to a pouch lining tissue that is morphologically like the synovium in joints, in which injection of sterile-filtered air under the skin on the back of the test animal provides an environment that is used to simulate the effects of diseases such as gout. As shown in FIG. 8, MSU crystals induced a robust increase in infiltrating inflammatory cells (mostly neutrophils and monocytes), which was significantly inhibited by either NR or NRH (p=0.0025 and p=0.007, respectively), indicating the capability of NR and NRH in reducing acute gouty inflammation. Given the potential of Ulodesine in lowering uric acid and of NR or NRH in limiting MSU crystal-induced inflammation, combination of NR or NRH with Ulodesine, or other PNP inhibitors, presents a novel therapeutic approach for gout.

### REFERENCES

1. Wielgus-Kutrowska B, Kulikowska E, Wierzchowski J, Bzowska A, Shugar D. Nicotinamide riboside, an unusual, non-typical, substrate of purified purine-nucleoside phosphorylases. Eur J Biochem. 1997; 243:408-14.
2. Hayat F, Migaud M E. Nicotinamide riboside-amino acid conjugates that are stable to purine nucleoside phosphorylase. Org Biomol Chem. 2020; 18:2877-2885.
3. Kropotov A, Kulikova V, Solovjeva L, Yakimov A, Nerinovski K, Svetlova M, Sudnitsyna J, Plusnina A, Antipova M, Khodorkovskiy M, Migaud M E, Gambaryan S, Ziegler M, Nikiforov A. Purine nucleoside phosphorylase controls nicotinamide riboside metabolism in mammalian cells. J Biol Chem. 2022; 298:102615.
4. Giroud-Gerbetant J, Joffraud M, Giner M P, Cercillieux A, Bartova S, Makarov M V, Zapata-Perez R, Sinchez-Garcia J L, Houtkooper R H, Migaud M E, Moco S, Canto C. A reduced form of nicotinamide riboside defines a new path for NAD+ biosynthesis and acts as an orally bioavailable NAD⁺ precursor. Mol Metab. 2019; 30:192-202.
5. Trammell S A, Schmidt M S, Weidemann B J, Redpath P, Jaksch F, Dellinger R W, Li Z, Abel E D, Migaud M E, Brenner C. Nicotinamide riboside is uniquely and orally bioavailable in mice and humans. Nat Commun. 2016; 7:12948.
6. Airhart S E, Shireman L M, Risler L J, Anderson G D, Nagana Gowda G A, Raftery D, Tian R, Shen D D, O'Brien K D. An open-label, non-randomized study of the pharmacokinetics of the nutritional supplement nicotinamide riboside (NR) and its effects on blood NAD⁺ levels in healthy volunteers. PLoS One. 2017;12:e0186459.
7. Martens C R, Denman B A, Mazzo M R, Armstrong M L, Reisdorph N, McQueen M B, Chonchol M, Seals D R. Chronic nicotinamide riboside supplementation is well-tolerated and elevates NAD(+) in healthy middle-aged and older adults. Nat Commun. 2018; 9:1286.
8. Conze D, Brenner C, Kruger C L. Safety and Metabolism of Long-term Administration of NIAGEN (Nicotinamide Riboside Chloride) in a Randomized, Double-Blind, Placebo-controlled Clinical Trial of Healthy Overweight Adults. Sci Rep. 2019 Jul. 5; 9(1):9772.
9. Dellinger R W, Santos S R, Morris M, Evans M, Alminana D, Guarente L, Marcotulli E. Repeat dose NRPT (nicotinamide riboside and pterostilbene) increases NAD⁺ levels in humans safely and sustainably: a randomized, double-blind, placebo-controlled study. NPJ Aging Mech Dis. 2017 Nov. 24; 3:17.
10. Simic P, Vela Parada X F, Parikh S M, Dellinger R, Guarente L P, Rhee E P. Nicotinamide riboside with pterostilbene (NRPT) increases NAD⁺ in patients with acute kidney injury (AKI): a randomized, double-blind, placebo-controlled, stepwise safety study of escalating doses of NRPT in patients with AKI. BMC Nephrol. 2020 Aug. 13; 21(1):342.
11. Fitz-Patrick D, Drummond W, Pappas J, et al. Effects of a purine nucleoside phosphorylase inhibitor, Bcx4208, on the serum uric acid concentrations in patients with gout[abstract]. Arthritis Rheumatism. 2010;62(Suppl 10):150.
12. Alan S. Hollister, Andreas Maetzell, Michael A. Becker, Robert Terkeltaub, David Fitz-Patrick, Valerie Smith and William P. Sheridan. Ulodesine (BCX4208) Long-Term Safety When Added to Allopurinol in the Chronic Management of Gout: A Phase 2 24-Week Blinded Safety Extension and Vaccine Challenge Study. 2012 ACR/ARHP Annual Meeting; Abstract No. 150.

What is claimed is:

1. A method of treatment for osteoarthritis or cartilage injury comprising administering to a subject in need thereof an effective amount of dihydronicotinamide riboside (NRH).

2. The method of claim 1, wherein the treatment increases NAD levels in articular chondrocytes of the subject.

3. The method of claim 1, wherein the treatment inhibits expression of matrix-degrading enzymes.

4. The method of claim 1, wherein the treatment inhibits progression of osteoarthritis or cartilage injury.

5. The method of claim 1, further comprising administering to the subject an effective amount of a purine nucleoside phosphorylase (PNP) inhibitor.

6. The method of claim 5, wherein the PNP inhibitor administration occurs before the NRH administration.

7. The method of claim 1, wherein the PNP inhibitor is forodesine or ulodesine.

8. A method of treatment for gout in a subject comprising administering to a subject in need thereof an effective amount of a purine nucleoside phosphorylase (PNP) inhibitor, and
administering to the subject an effective amount of dihydronicotinamide riboside (NRH).

9. The method of claim 8, wherein the PNP inhibitor administration occurs before the NRH administration.

10. The method of claim 8, wherein the PNP inhibitor is forodesine or ulodesine.

11. The method of claim 8, wherein the treatment increases NAD levels in the subject.

12. The method of claim 8, wherein the treatment inhibits expression of matrix-degrading enzymes.

13. The method of claim 8, wherein the treatment inhibits progression of gout.

14. A method of treatment for aging-associated disease comprising administering to a subject in need thereof an effective amount of a purine nucleoside phosphorylase (PNP) inhibitor, and
administering to the subject an effective amount of dihydronicotinamide riboside (NRH).

15. The method of claim 14, wherein the PNP inhibitor administration occurs before the NRH administration.

16. The method of claim 14, wherein the PNP inhibitor is forodesine or ulodesine.

17. The method of claim 14, wherein the aging-associated disease is selected from cardiovascular disease, neural and cognitive function disease, metabolic disease, muscular disease and kidney disease.

18. The method of claim 14, wherein the treatment increases NAD levels in the subject.

19. The method of claim 14, wherein the treatment inhibits expression of matrix-degrading enzymes.

20. The method of claim 14, wherein the treatment inhibits progression of aging-associated disease.
